(19) United States
(12) Patent Application Publication
(10) Pub. No.: US 2004/0266992 A1
(43) Pub. Date: Dec. 30, 2004

(54) Method for the preparation of silk fibron hydrogels

(76) Inventors: Claudio Migliaresi, Trento (IT); Kemal Kesenci, Yenliman-Trototo Antalya (TR); Antonella Motta, Milano (IT)

Correspondence Address: Charles Berman, Greenberg Traurig, 2450 Colorado Avenue, Suite 400E, Santa Monica, CA, 90404, US

(21) Appl. No.: 10/489,192

(22) PCT Filed: Sep. 11, 2002
(86) PCT No.: PCT/IT02/00579

(30) Foreign Application Priority Data
Sep. 11, 2001 (IT) VR2001A000098

(51) Int. Cl. A61K 38/17; A61K 9/14; C07K 14/435

(52) U.S. Cl. 530/353; 424/488

(57) ABSTRACT

Process for the production of silk fibroin hydrogels comprising the following steps: a) Obtaining the raw silk filament for example from cocoons, silk fabric, raw silk waste; b) Removing where present the sericin layer covering the silk fibroin fibers; c) obtaining a solution of fibroin in water; d) Treating the fibroin solution with water soluble polymers and/or with acid solutions with a pH lower than 4 and/or polar solvents and/or with crosslinking compounds. e) Washing the resulting material from said step b and so obtaining a silk fibroin hydrogel.

### TECHNICAL FIELD

The present invention refers to a process for the preparation of silk fibroin hydrogels, particularly for silk produced from silk worm (Bombyx Mori).

Particularly the invention relates to a process for the production of silk fibroin hydrogels suitable to be used as bio-material for implants in surgical operations and for the reconstruction of both soft and hard tissues, correction of soft and hard tissues defects and for bio-material aiding the wound healing.

Further applications comprise the use of the silk fibroin hydrogels as scaffolds for cell cultures applicable in tissue engineering and cell biology, as immobilization and controlled release matrix for drugs, biologically active compounds or for their association.

Silk fibroin hydrogels according to the present invention can also be used as coating for implants or other devices in order to improve their biocompatibility and/or cell and tissue response.

### BACKGROUND ART

Hydrogels are semisolid forms made of polymeric compounds, which can have both natural and synthetic origin and are characterized from the ability to incorporate considerable amounts of water with reference to their initial mass weight.

The physicochemical characteristics of the hydrogels depend from the grade and the nature of the bonds present inside the polymeric structure, from its grade of crystallinity, from the interactions between the molecules of the polymer and those of the solvent, the salts present therein or the chemical compounds adsorbed on the gel.

Other fundamental factor regulating the stability and the characteristics of the gel are the aging, the temperature, the pH, the solvent polarity, the salt present therein and the formation of possible complexes.

A possible change of one of the factors mentioned above modifies the characteristics of the gel, which can lose its typical state: in this case it is said that the gel collapses.

The occurring modifications can be reversible or irreversible depending on the nature of the material and on the modified parameter.

The balance between the hydrophobic and hydrophilic groups of the gel is the key factor regulating its characteristics and which allows its use as an innovative material in different sectors.

The essential characteristics required to an hydrogel, which have a key role in relation to its use in the biomedical field, are the biocompatibility, the non-immunogenicity, the simple applicability, the ability to maintain the structure and the physicochemical characteristics in a physiological environment as well as the possibility to be degraded and/or metabolized.

The ability to keep or eventually release in a second time different substances, such as for example pharmaceutical compounds or biologically active compounds without changing their activity or causing their degradation, is critical when the gel is used as a scaffold.

Between the several applications in which hydrogels show a clear interest there is their use for the release of biologically active compounds, for example growing factors, in application of tissue engineering and cell biology, as well as for coating or implants systems or other devices, as excipients or vehicles for pharmaceutical formulations and simply as filler material to repair or correct tissues deficiencies.

The main advantage of several natural hydrogels compared to the synthetic one is their biocompatibility as well as the biocompatibility of their secondary derivatives eventually released.

In relation to this characteristic the silk fibroin can be a material for the production of natural hydrogels with a great biocompatibility and non-immunogenicity.

Fibroin is a fibrous protein obtained from the silk of silk worm (Bombix Mori) and constitutes about the 75 to the 80% by weight of the silk at the raw state.

The silk comprises also between 20 and 25% by weight of another protein, the sericin, which cover the fibroin filaments and can be removed by means of a degumming process.

With reference to the secondary function of the fibroin, the β-sheet structure is responsible for the gelation process (Hirabayashi, K., Ayub, Z. H., and Kume, Y., Sen-i Gakkaishi, 1990, 46, 521) and the formation of said structure would dependent from aging, temperature, pH, polar solvents and complex formation as already mentioned above.

The use of natural hydrogels is already well reported in the literature.

Hydrogels are used for the production of soft lenses, as well as in the tissue-engineering field for soft tissue materials replacement (Chvapil, M., Kronenthal, R. L., Winkel, W. V., In: Hall, D. A., Jackson, D. S., editors. Medical and surgical applications of collagen. International Review of Connective Tissue Research, vol. 6. New York: Academic Press, 1973.p.1.), or for drug release carriers.

A process for producing silk fibroin hydrogels suitable for use as artificial muscles or artificial blood vessel walls is described in the Japanese Patent No: JP1308431.

This process uses fibroin from directly obtained from silk of the silk glands of mature silkworms after removing the sericin fraction.

Silk fibroin produced with this process has low strength and therefore is unsuitable for several applications such as for implant material.

The article entitled: “New oral dosage form for elderly patients: preparation and characterization os silk fibroin gel” (Hanawa T et al Chem. Pharm. Bull. 43(2), 1995, 254-288) describes the pharmaceutical utility of silk fibroin as a possible material for an oral dosage form. The preparation of silk fibroin gel is summarized in the flow-charts 1 and 2 and explained in detail in the experimental part of the paper.

The preparation comprises a first stage leading to the production of silk fibroin powder and a second stage using silk fibroin powder as starting material for the final production of the fibroin hydrogel. This is a clear drawback in view the whole process for the production of a silk fibroin hydrogel.

JP-A-5604156 describes a method to prepare a titled material having specific average degree of polymerization, average particle diameter and bulk density, and useful as a raw material of cosmetic. Said material is economically producible, in a industrial scale, by dissolving glossed silk in a specific aqueous medium, dialyzing and hydrolyzing the solution, and spray-drying to obtain powdery material. Also in this case with the aim to obtain a silk fibroin hydrogel the presence in the process previously described of a spray-draying phase is a drawback.

JP-A-01254164 discloses a wound protective material which is constituted of the porous body of the silk fibroin gel to improve the fittability of such a material to a living body, the state of adhesion to the wound and the retentivity of the adhesion. The wound protective material is produced by casting an aqueous silk fibroin solution into a molding flask as it is or after addition of a treating agent thereto and drying the molding or gelling the solution, forming the gel into a film shape. JP-A-01254164 describes only the possibility of pH adjustment for gelling the solution.

JP-A-01254621 describes a drug carrier composed of a porous silk fibroin, capable of stably keeping a drug in the cavities of a three-dimensional network structure. A fibroin aqueous solution is freeze-dried and pulverized to obtain silk powder or is cast in the form of a film to obtain a powdery or filmy drug carrier. Freeze-drying is a further procedural step which is not essential for the hydrogel preparation.

JP-A-01118544 refers to a porous product of silk fibroin which can be easily formed with various pore diameters in a lyophilized gel form. Said product can be produced by forming water-insoluble gel from an aqueous solution of silk fibroin, forming the obtained gel-like substance as a molding material into a porous product of any desired shape. The pore size can be controlled by the concentration of the silk fibroin solution, the pH conditions for gelation, the kind and concentration of a poor solvent used for gelation, the lyophilization temperature.

U.S. Pat. No. 4,264,493 discloses natural protein hydrogel structures formed from natural proteins having molecular weights not exceeding 100,000 by dissolving the protein in an aqueous acidic solution, crosslinking the protein, and air drying to a moisture content not exceeding 10 percent. Furthermore, according to U.S. Pat. No. 4,264,493 (column 2, lines 12-14) it is essential to begin with natural protein raw materials that form clear solutions in water at concentrations up to 30 percent or higher.

Silk fibroin has a molecular weight of about 370,000 and a solubility in water much lower than 30 percent.

### DESCRIPTION OF THE INVENTION

The main scope of the present invention is to provide a process for the production of silk fibroin hydrogels produced from silk worm (Bombyx Mori), having characterizing physicochemical properties, non-immunogenic activity and a high biocompatibility.

The main scope previously reported is achieved with the features disclosed in the main claim.

The dependent claims outline particularly advantageous forms of embodiment of the procedure according to the invention.

Furthermore, claims 20 and 21 describe a silk fibroin hydrogel.

Moreover, claims 22, 23 and 24 describe particularly advantageous uses of silk fibroin gels.

The process according to the present invention comprises the initial preparation of an aqueous solution of silk fibroin obtained for example from cocoons, raw silk waste, silk fabric waste.

Prior to use, raw silk is degummed by washing in boiled water containing a surface-active agents like for example sodium dodecylsulfate (SDS), sodium carbonate (Na₂CO₃), calcium carbonate (CaCO₃), or with an enzyme solution such as papain or chemotrypsin. It is preferable to degum the silk fibroin with sodium dodecylsulfate and sodium carbonate. Said methods are also well known in the art and described in scientific literature.

Afterwards, the so purified fibroin fibers are treated in such a way to obtain a final aqueous salt solution of the protein in the range of about 1% to 20% by weight.

According to a form of embodiment of the invention, the aqueous salt solution, used for the preparation of the fibroin solution, favorably comprises a mixture of salts chosen in the raw of lithium bromide, calcium chloride, zinc chloride, magnesium chloride, lithium thiocyanate and sodium thiocyanate with a concentration in the range of about 40% to 60% by weight.

The dissolution process of the fibroin is usually performed at a temperature in the range of about 20 to 60° C.

The process according to present invention comprises that the salt present in the silk fibroin solution is removed. Preferably, this is obtained by dialysis using semi-permeable membranes typically made of cellulose.

Forms of embodiment of the present invention comprise for example, the production of fibroin hydrogels by means of dialysis treatment of a fibroin solution against water soluble polymer, acids, polar solvents or solutions containing crosslinkers,

The dialysis process is performed until the silk fibroin precipitates.

The obtainment of silk fibroin gels according to the invention can be achieved with different processes, as those reported in the following:

By means of water soluble polimers such as, for example, polyethylene glycol, polyvinyl alcohol, polyvinyl pyrolidone, or polyacrylic acid solutions. The fibroin solution is treated with a dialysis process with a solution of more water-soluble polymers. Due to molecular interaction between silk fibroin chains and the polymer the hydrogel formation is obtained.

By means of acid solution like for example concentrated sulfuric acid solution or other acids such as nitric acid, phosphoric acid or acetic acid in water. Therefore, a silk fibroin solution is dialyzed with an acid solution until a pH lower than 4 is reached, in so doing a pH lower than the isoelectric point of the silk fibroin, which correspond to pH of about 4.7, is achieved allowing the formation of new weak- and hydrogen-bonds determining the formation of the gel.

By means of water miscible polar solvents such as for example methanol, ethanol, ethyl acetate, isopropanol. The gel formation is obtained for example through a process of dialysis of the silk fibroin solution inside a semi-permeable conteiner such as a cellulose tube, with a methanol water solution.

By means of crosslinkers such as glutaraldehyde or epichlorohydrin water solutions.

Said compounds allow the formation of inter- and intra-molecular bonds through reactions between hydroxyl and amine groups of the protein, as well as formation of new hydrogen bonds or other non-covalent bonds which result in the silk fibroin hydrogels formation.

Silk fibroin was proved to be biocompatible, non-immunogenic material and therefore not interfering with the normal cell mediated immune response. Another advantage of the silk fibroin is its favorable mitogenic action, as reported in the Italian patent VR2000A000096, particularly for keratinocytes, fibroblasts, and human endothelial cells.

Silk fibroin hydrogels obtained from silk of silk-worm (Bombyx Mori) according to the invention can be realized with a wide range of physicochemical characteristics and are characterized by an open pore structure which allows their use as tissue engineering scaffolds, substrate for cell culture, wound and burn dressing, soft tissue substitutes, bone filler, and as well as support for pharmaceutical or biologically active compounds.

### ILLUSTRATION OF DRAWINGS

Other features and advantages of the invention will become apparent by reading the following description with the help of the figures illustrated in the attached drawings. The following forms of embodiment are given as non-limiting examples of the invention.

FIG. 1 is a representative optical micrograph of a silk fibroin hydrogel sample as prepared (at the right) and dried (at the left).

FIG. 2 shows two photographs taken with an Environmental Scanning Electron Microscope (ESEM®) of a silk fibroin hydrogel completely hydrated (sample on left—A) and after the beginning of the water loss stage (sample on the right—B); the photos in FIG. 2 were taken with an Environmental Scanning Electron Microscope (ESEM®), Philips, with not dehydrated and uncoated samples.

FIG. 3 shows a graphic related to the water loss rate of silk fibroin hydrogels, as a function of time, produced with process using water soluble polymer (A), Sulfuric acid (B), Methanol (C), and Glutaraldehyde (D).

The water loss of the silk fibroin hydrogels was calculated by using the following equation:

Water Loss Percent (%)=(1−(Ms/Md))*100 (1).

Where; Ms is the mass of swollen silk fibroin hydro gels (g) and Md is dry mass hydrogels (g)

FIG. 4 shows the results obtained by thermogravimetric analysis of silk fibroin hydrogels samples obtained using the process with water soluble polymer (A), Sulfuric acid (B), Methanol (C), and Glutaraldehyde (D).

Analysis were performed under a nitrogen flux of 100 ml/min. and with a heating rate of 10° C./min. in a range of temperature from 30 to 450° C. sufficient to volatile the sample.

A thermogravimetric analyser (Mettler TG50) was used for the measurements. With such analysis the water content and thermal degradation behavior of the samples were evaluated.

FIG. 5 shows the results obtained by differential scanning calorimetry analysis (DSC) of silk fibroin hydrogels samples obtained using the process with water soluble polymer (A), Sulfuric acid (B), Methanol (C), and Glutaraldehyde (D).

Analysis were performed under a nitrogen flux of 100 ml/min. and with a heating rate of 10° C./min. in a range of temperature from 20 to 320° C. using an instrument Mettler, Model DSC30.

FIG. 6 shows typical infrared absorption spectra of silk fibroin hydrogels prepared respectively using the process with water soluble polymer (A), Sulfuric acid (B), Methanol (C), and Glutaraldehyde (D) in their wet state (graphics A)

### DESCRIPTION OF SAME FORMS OF EMBODIMENT

As previously mentioned, silk cocoons, silk fabric material, or raw silk waste can be used with the aim to obtain silk fibroin.

Furthermore, since the row silk filament comprises an external layer of sericin, the silk has first to be treated to remove said sericin layer.

The degumming process can be any of those known in the art and described in the scientific literature such as according to the present form of embodiment of the invention, by means of a treatment with a warm sodium carbonate (Na₂CO₃) and sodium dodecylsulfate (SDS) solution.

According to the invention, the degummed silk is then dissolved in an aqueous lithium bromide solution (LiBr) or in other salt solutions between those known in the art, finally obtaining a clear, thick solution of silk fibroin with a yellowish color.

At this point, the salt in the solution is removed by means of a dialysis process having different passages in water in a time range of more than 20 hours; finally a water solution of silk fibroin with a concentration of about 10% by weight is obtained.

The characterization of the fibroin gel was performed using an Environmental Scanning Electron Microscope (ESEM®), thermogravimetric analysis (TGA) and differential scanning calorimetry analysis (DSC), Infrared spectrophotometry, and mechanical tests as well.

Examples of some forms of embodiment of hydrogels according the present invention are reported in the following.

### EXAMPLE 1

An aqueous solution of silk fibroin prepared by dialysis as reported above, still kept in the cellulose dialyzing tube, is immersed in a 50% by weight polyethylene glycol (Mw: 1000)/water solution. In the course of 2-3 hours, gentle stirred, the silk fibroin gradually precipitates in the cellulose tube forming, at the end of the process, a jellified mass. At this point, the silk fibroin hydrogel is taken out from the cellulose tube, washed and stored in distilled water.

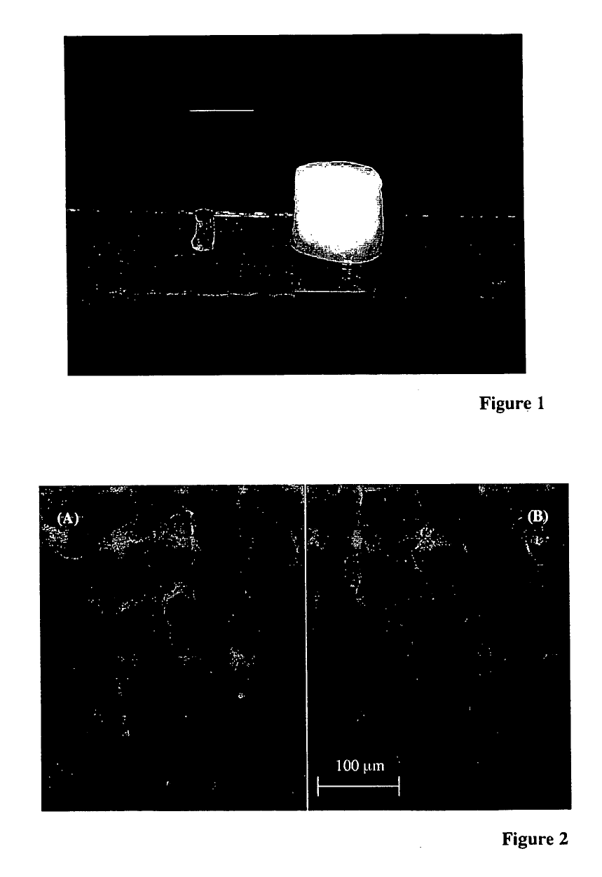

When the water loss was determined according to the above-described procedures, it was found that the silk fibroin hydrogel shrank on dehydration, with a corresponding change in color (FIG. 1), from its initial white opaque color to yellowish.

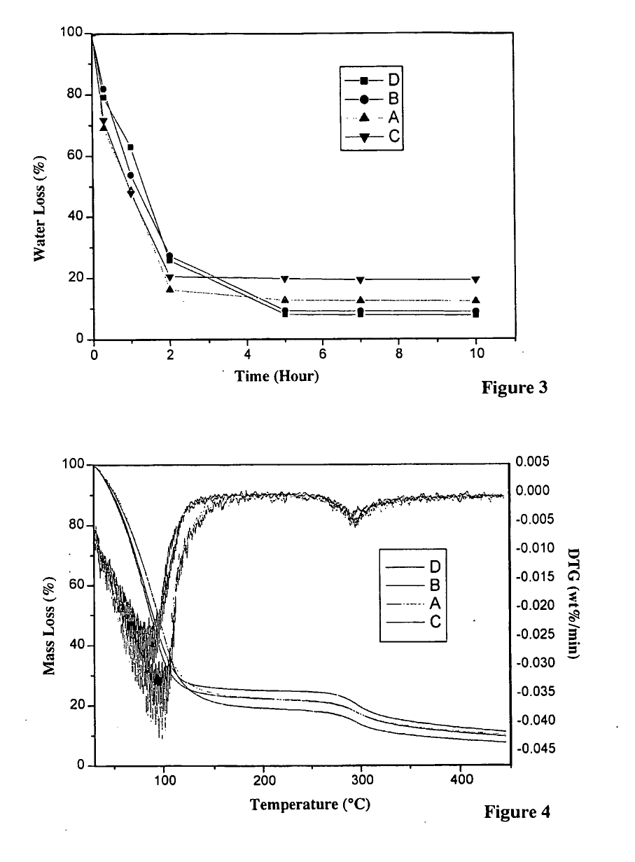

The hydrogel displays homogenous structure, having an apparent pore size in the swollen form ranging in the field of 50 to 100 μm (FIG. 2). The water loss percentage of the hydrogel stored outside in the air is approximately 87%-wt (FIG. 3A).

The silk fibroin hydrogel undergoes two main thermal decomposition stages: the first one is an abrupt water evaporation from silk fibroin molecules which occurs from 50 to 150° C.; the second one, a decomposition from 250 to 350° C.

The weight loss value of 87%-wt inferred by TGA, as the hydrogel is heated beyond 120° C., supports identification of vaporization of water associated with the drying of silk fibroin hydrogel (FIG. 4A).

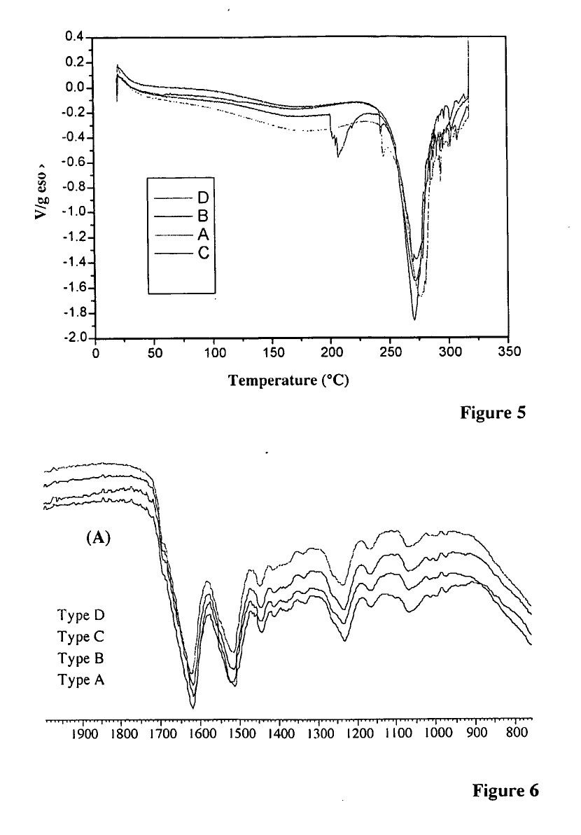

The temperature of the peak of the degradation of the silk fibroin was 294° C. Silk fibroin hydrogels, depending on the preparation process, could also exhibit a lower temperature degradation peak at 273° C. (FIG. 5A).

Conformational characterization of silk fibroin hydrogels was carried out by means of Fourier transformed infrared spectroscopy FTIR (FIG. 6A).

TABLE 1
Characteristic FTIR bands of silk fibroin hydrogels.

| Hydrogel | | CH3 and | | | |
|---|---|---|---|---|---|
| Type | OH* | CH2 | Amide I | Amide II | Amide II |
| Type A | 3500 | 3277, 2932 | 1620 | 1510 | 1227 |
| Type B | 3500 | 3277, 2932 | 1655, | 1541, 1508, | 1225 |
| | | | 1636, 1618 | 1499 | |
| Type C | 3500 | 3277, 2932 | 1620 | 1510 | 1227 |
| Type D | 3500 | 3277-2932 | 1618 | 1510 | 1227 |

*Free OH coming from water was only observed in wet samples.

The samples were dehydrated and directly examined by a FTIR spectrophotometer (Bio-Rad, FTS165, USA).

As shown in Table 1, the spectral region 3000-2800 cm⁻¹ is characteristic of the primary structure of carbon backbone of polypeptide coming from anti symmetric and symmetric CH₃ or CH₂ vibrations.

The absorption at 1618 cm⁻¹ occurs in a region that is characteristic for anti-parallel β-sheet, while 1655 cm⁻¹ and 1685 cm⁻¹ belong to β-turns and bends of the secondary structure of the silk fibroin.

TABLE 2
Shore A values for wet silk fibroin hydrogels.

| Hydrogel Type | Shore A (load grams) |
|---|---|
| Type A | 10 ± 3 |
| Type B | 5 ± 1 |
| Type C | 20 ± 5 |
| Type D | 15 ± 3 |

Hardness was measured with an ATS Faar S.p.A., according to the standard ASTM D 2240.

Table 2 reports the hardness of wet silk fibroin hydrogels.

### EXAMPLE 2

An aqueous solution of silk fibroin was prepared by dialyzing the silk/lithium bromide solution against distilled water over a 24 h period as previously described. The so prepared aqueous solution was kept in a dialyzing tube and immersed in a 0.1 N sulfuric acid solution.

Then, the obtained silk fibroin hydrogel was taken out from the cellulose tube, washed several times and stored in distilled water.

This hydrogel also displayed a homogenous pore structure, with the pore size ranging when swollen in the field from 50 to 100 μm. The final water loss at atmospheric conditions was approximately 92% (FIG. 3B). Thermal decomposition occurred at 300° C. (FIG. 4B). Temperatures of the endothermic peaks were 212° C. and 273° C. (FIG. 5B).

The wet silk fibroin hydrogels had 5±1 hardness shore A values.

### EXAMPLE 3

An aqueous solution of silk fibroin was prepared by dialyzing the silk/lithium bromide solution against distilled water over a 24 h period as previously described. Then, the aqueous solution of silk fibroin was kept in the semi-permeable cellulose tube and immersed in an 80%-wt methanol/water solution.

In the course of 2 to 3 hours, keeping the solution under gentle agitation, the silk fibroin gradually precipitated in the cellulose tubes forming, at the end of the process, a jellified mass. At this point, the silk fibroin hydrogel was taken out from cellulose tube, washed with water and kept in distilled water.

This hydrogel also displayed homogenous structure, with pores sizing from 50 to 100 μm. The hydrogel final water loss percentage at atmospheric conditions was approximately 80%-wt (FIG. 3C). The silk fibroin hydrogel exhibited a sharp endothermic peak at 275° C. (FIG. 5C). Wet silk fibroin hydrogels had 20±5 shore A hardness values.

### EXAMPLE 4

An aqueous solution of silk fibroin was prepared by dialyzing the silk/lithium bromide solution against distilled water over a 24 h period as previously described. The aqueous solution of silk fibroin so prepared was kept in semi-permeable cellulose tube and immersed in a 20%-wt glutaraldehyde/water solution.

In the course of 2 to 3 hours after gentle agitation, the silk fibroin gradually precipitated in the cellulose tubes forming, at the end of the process, a jellified mass. Then, the silk fibroin hydrogel was taken out from the cellulose tube, washed and kept in distilled water.

The final water loss percentage of the hydrogel at atmospheric conditions was approximately 90%-wt (FIG. 3D). This silk fibroin hydrogel exhibited sharp endothermic peaks at 245° C. and 278° C. (FIG. 5D) and thermal decomposition at 290° C. (FIG. 4D). The wet silk fibroin hydrogel had 15±3 shore A hardness values.

The previous description states that the silk fibroin hydrogels, according to the invention, thanks to their physicochemical properties and their particular structure can be used as tissue engineering scaffolds, substrate for cell culture, wound and burn dressing, soft tissue substitutes, bone filler, and support for pharmaceutical or biologically active compounds.

The invention has previously been described with reference to some preferred forms of embodiment of the same.

However it is clear that the present invention is susceptible to several modifications and variations within the scope of the present invention as it was defined in the appended claims.

For example, on the base of the model previously exposed, other silk fibroin gels can be provided of specific features for their use as bio-materials for implants in surgical operations and for the reconstruction of both soft and hard tissues, repair of soft and hard tissues defects and for bio-material supporting the wound healing.

Further applications comprise the use of the silk fibroin hydrogels as scaffolds for cell cultures applicable in tissue engineering and cell biology, as immobilization and controlled release matrix for drugs, of biologically active compounds or for their association.

The silk fibroin hydrogel according to the present invention can be used as covering material for implant systems or for other system to improve the biocompatibility and/or the tissue and cell response.

1. Process for the production of silk fibroin hydrogels comprising the following subsequent steps:
a) Obtaining the raw silk filament for example from cocoons, silk fabric, raw silk waste;
b) Removing where present the sericin layer covering the silk fibroin fibers by means of a treatment with a warm sodium carbonate (Na2CO3) and sodium dodecylsulfate (SDS) solution or by means of other degumming method known in the art;
characterized by the following steps:
c) Obtaining a solution of fibroin in water by dissolving the silk fibroin in a water salt solution and following elimination of the salt by dialysis;
d) Treating the fibroin solution with water soluble polymers and/or with acid solutions with a pH lower than 4 and/or polar solvents and/or with crosslinking compounds.
e) Washing the resulting material from said step b and so obtaining a silk fibroin hydrogel.

2. Process according to claim 1 in which the water solution of said step c comprises an amount of a salt mixture in the range of about 0.1 to 10% by weight said salt selected in the row of calcium chloride, zinc chloride, potassium chloride, lithium bromide, lithium thiocianate, magnesium chloride, copper nitrate and sodium chloride.

3. Process according to claim 2, in which for the dissolution of the silk fibroin is used a solution of lithium bromide (LiBr)

4. Process according to claim 2 in which the silk fibroin is comprised in range of about 1 to 20% by weight of said solution.

5. Process according to claim 4 in which step c is performed at a temperature not higher than 60° C.

6. Process according to anyone to claim 4 in which said step c is performed at room temperature.

7. Process according to claim 1 in which said step d is performed treating the fibroin solution with a solution of water soluble polymer of the group comprising polyethylene glycol, polyvinyl alcohol, polyvinyl pyrolidone, or polyacrylic acid or with a solution containing one their resulting mixture.

8. Process according to claim 7 in which a solution of polyethylene glycol at a concentration of 50% by weight is used.

9. Process according to claim 1 in which said step d is performed treating the fibroin solution with an acid solution in such a way to obtain a pH value lower than 4.

10. Process according to claim 9 in which the acid solution is a water solution of one of the acid or one of their mixture comprising sulfuric acid, nitric acid, phosphoric acid or acetic acid

11. Process according to claim 10 in which the acid solution is a water solution of sulfuric acid 0.1N.

12. Process according to claim 1 in which said step d is performed treating the fibroin solution with a solution of crosslikers or with a solution comprising a mixture of such consslinkers such as glutaraldehyde or epichlorohydrin.

13. Process according to claim 12 in which a water solution of glutaraldehyde at a concentration of 20% by weight is used.

14. Process according to claim 1 in which said step d is performed treating the fibroin solution with water miscible polar solvents or with a mixture of them comprising methanol, ethanol, ethyl acetate, isopropanol

15. Process according to claim 14 in which a methanol water solution at concentration of about 80% by weight is used.

16. Process according to claim 1 preformed by means of a dialysis process.

17. Process according to claim 1 in which said step c refers to wash repeatedly the material with distilled water.

18. Silk fibroin hydrogel characterized in that it comprises a homogenous structure, having open pores with an apparent size ranging in the field of 50 to 100 μm.

19. Tissue according to claim 18 characterized in that is obtained by means of a process according claim 1.

20. Use of a silk fibroin gel according to claim 19 for as bio-materials for implants in surgical operations and for the reconstruction of both soft and hard tissues, repair of soft and hard tissues defects and for bio-material supporting the wound healing.

21. Use of variants of the gel according to claim 18 commercially provided as scaffolds for cell cultures applicable in tissue engineering and cell biology, as immobilization- and controlled release matrix for drugs, of biologically active compounds and/or for their association.

22. Use of variants of the gel according to 18 commercially provided as covering material for implant systems or for other system with the a to improve the biocompatibility and/or the tissue and cell response.

23. Use of a silk fibroin gel according to claim 20 for as bio-materials for implants in surgical operations and for the reconstruction of both soft and hard tissues, repair of soft and hard tissues defects and for bio-material supporting the wound healing.

24. Use of variants of the gel according to claim 19 commercially provided as scaffolds for cell cultures applicable in tissue engineering and cell biology, as immobilization and controlled release matrix for drugs, of biologically active compounds and/or for their association.

25. Use of variants of the gel according to claim 20 commercially provided as scaffolds for cell cultures applicable in tissue engineering and cell biology, as immobilization and controlled release matrix for drugs, of biologically active compounds and/or for their association.

26. Use of variants of the gel according to claim 19 commercially provided as covering material for implant systems or for other system with the aim to improve the biocompatibility and/or the tissue and cell response.

27. Use of variants of the gel according to claim 20 commercially provided as covering material for implant systems or for other system with the aim to improve the biocompatibility and/or the tissue and cell response.

28. Use of variants of the gel according to claim 21 commercially provided as covering material for implant systems or for other system with the aim to improve the biocompatibility and/or the tissue and cell response.
